(19) United States
(12) Patent Application Publication
(10) Pub. No.: US 2020/0130031 A1
(43) Pub. Date: Apr. 30, 2020

(54) Automatic sterilizing waste glass bottle recycling equipment

(71) Applicant: Shichang Lin, Fuzhou City (CN)

(72) Inventor: Shichang Lin, Fuzhou City (CN)

(21) Appl. No.: 16/726,195

(22) Filed: Dec. 23, 2019

(30) Foreign Application Priority Data
Sep. 19, 2019 (CN) 2019108858526

(51) Int. Cl. B08B 9/42; C03C 23/00; B09B 5/00; B08B 9/08; B08B 9/087; B08B 9/093; B08B 9/46; B08B 9/30; B08B 9/36; B08B 9/38; A61L 2/26; A61L 2/18

(52) U.S. Cl. CPC B08B 9/42; A61L 2202/14; B09B 5/00; B08B 9/083; B08B 9/087; B08B 9/093; B08B 9/0826; B08B 9/46; B08B 9/30; B08B 9/36; B08B 9/38; A61L 2/26; A61L 2/18; B08B 2203/007; B08B 2209/08; B08B 2220/01; A61L 2202/23; A61L 2202/17; C03C 23/0075

(57) ABSTRACT

The invention discloses an automatic sterilizing waste glass bottle recycling equipment, and includes a machine body, a left side of the upper end of the machine body is provided with a placement opening that opens upwards and opens to the left, a right wall of the placement opening communicates with a channel that opens to the right, and a bottle washing device is provided on the left side of the channel An infrared detection device is arranged in the channel and on the right side of the bottle body scrubbing device, the infrared detection device pushes out bottles with foreign objects or damage in the bottle, and an in-bottle flushing device is provided on the right side in the channel. The outer surface of the recovered glass bottle can be quickly cleaned and the old labels can be removed. By hot water cleaning, both the bottle body can be sterilized by high temperature and the glass bottle can be heated at the same time, so that the next infrared detection can be accurately removed.

### CROSS-REFERENCES TO RELATED APPLICATIONS

The present application claims priority from Chinese application No. 2019108858526 filed on Sep. 19, 2019 which is hereby incorporated by reference in its entirety.

### FIELD OF TECHNOLOGY

The invention relates to the technical field of bottle washing machine equipment, and in particular relates to an automatic sterilizing waste glass bottle recycling equipment.

### TECHNICAL FIELD

Glass bottles of beer and beverages account for a large share of beverage sales. Compared to cans, glass bottles can be recycled at a lower cost, which can save more costs for enterprises. Most of the cleaning and disinfection work is a large-scale assembly line composed of devices with separate functions. The equipment has a complicated structure, high purchase and maintenance costs, and a large floor area. For some small and medium-sized enterprises, the entry and operating costs are too high. The present invention illustrates a device capable of solving the above problems.

### CONTENT OF THE INVENTION

Technical problem: the current work for glass bottle recycling and cleaning is completed by the assembly of separate functions into a large-scale assembly line. The equipment structure is complex, the purchase and maintenance costs are high, and the area is large. For some small and medium enterprises, the entry and operating costs are too high.

In order to solve the above problem, this example designs a waste glass bottle recycling device that can be automatically sterilized. A waste glass bottle recycling device that can be automatically sterilized in this example includes a machine body, and the left end of the upper end of the machine body is provided with an opening upward. And a left opening, the right wall of the opening is connected with a right passage, and the left side of the passage is provided with a bottle washing device, and the bottle washing device includes a symmetrical and communicating arrangement The brush roller cavity on the left side of the front and rear walls of the channel is provided with a rotating shaft uniformly and rotatably between the upper and lower walls of the channel. A brush roller is fixed on the outer surface of the rotating shaft, and the close end of the brush roller extends to In the channel, the label on the surface of the passing glass bottle is cleaned. An infrared detection device is provided in the channel and on the right side of the bottle washing device. The infrared detection device includes a communication provided on the rear wall of the channel and A bottle pushing chamber on the right side of the brush roller chamber is provided with a bottle pushing lever which is rotatably connected to the upper end of the right wall of the bottle pushing chamber, and the bottle pushing chamber is turned forward to turn the bottle The bottle with foreign objects or damage is pushed out, An in-bottle scouring device is provided on the right side of the channel, and the in-bottle scouring device includes a chute connected to the upper and lower walls and the front and rear walls of the channel. A cylinder is rotatably provided in the chute, and the center of the cylinder is provided. The left and right through holes are provided with the same shape as the channel. After the glass bottle enters the through hole, the bottle flushing device cleans and disinfects the bottle inside the through hole and blocks the left side of the bottle flushing device. Bottle moves to the right.

Preferably, the lower ends of the front and rear walls of the placement port are symmetrically and rotatably provided with a transmission shaft. A transmission wheel is fixed on the outer circular surface of the transmission shaft, and a transmission belt is wound around the transmission wheel. A first motor is fixed in the rear wall and behind the transmission shaft on the right side, the rear end of the transmission shaft on the right side is power-connected to the front end of the first motor, and the right wall of the placement port is fixed on the upper side. A splice plate whose end surface is located on the same plane as the lower wall of the channel, the left end of the splice plate extends to the right side of the conveyor belt, and the end surface of the splice plate is slightly lower than the upper end surface of the conveyor belt.

Wherein, the bottle body washing device includes a transmission cavity provided in the lower wall of the brush roller cavity and below the rotating shaft, the lower end of the rotating shaft extends into the driving cavity and a sprocket is fixedly arranged, and the chain A roller chain is arranged between the wheels, and a second motor is fixed in the lower wall of the transmission cavity and below the rightmost rotating shaft, and the lower end of the rightmost rotating shaft is dynamically connected to the first Second motor.

Preferably, a water tank is connected to the lower wall of the channel, and a filter plate whose upper end is located on the same plane as the lower wall of the channel is fixed on the upper end of the front and rear walls of the channel, and a first water pump is fixed on the lower wall of the channel A first spray head is evenly distributed and fixed on the rear wall of the channel and above the brush roller cavity, and a first water supply channel is provided between the first water pump and the first spray head. A scraper is fixed on the side of the wall or the rear wall and away from the center of symmetry of the rotation axis. The lower wall of the brush roller cavity is connected with a discharge port with an opposite opening direction under the scraper. The water pump sprays the water in the water tank from the first nozzle through the first water supply channel to flush the bottle body, the scraper scrapes off the labels washed and washed by the brush, and the labels slide from the outlet Out.

Wherein, the infrared detection device includes a third motor fixedly arranged at the lower end of the left wall of the bottle pushing cavity, a front end of the third motor is power-connected with a turntable, and a side of the front end surface of the turntable is rotatably provided A connecting rod, the other end of which is rotatably connected to the lower end of the bottle pushing rod.

Preferably, an electric heating wire is fixed between the left and right walls of the water tank and above the first water pump, and an electric connection to the third wall is fixed on the upper wall of the channel and above the push rod. An infrared generator of a motor, a channel on the front wall of the channel is opened in front of the bottle pushing rod, and a lower wall of the channel is inclined downward and forward and the rear end is higher than the lower wall of the channel. The electric heating wire heats the water in the water tank, and hot water is sprayed from the first spray head to the heated bottle, and the infrared generator detects the bottle below, and the bottle with foreign matter or damage is pushed to the bottle Inside the slide.

Wherein, the flushing device in the bottle includes a hydraulic arm fixedly arranged at the center of the upper wall of the port, and a lower end of the hydraulic arm is fixedly provided with a hanging plate, and the lower end surface of the hanging plate is uniformly distributed and fixed with a second spray head. When the hydraulic arm is extended to drive the hanging plate to drop against the bottle mouth, the second spray head is inserted into the bottle mouth.

Preferably, the left end surface of the cylinder is provided with a tooth slot opening to the left, a lower end of the left wall of the sliding slot is connected with a rotary power cavity, and a fourth motor is fixedly installed in the lower wall of the rotary power cavity. The upper end of the four motors is power-connected with a gear with a right end meshing with the cogging.

Preferably, a disinfection liquid cavity is provided in the lower wall of the chute, a second water pump is fixedly located at the lower end of the right wall of the disinfection liquid cavity, and a second water supply channel is connected to the right end of the second water pump. A semi-circular hose passage is connected to the right wall of the chute, and a hose with a lower end connected to the second water supply passage is provided in the hose passage. The upper end of the hose passes through the cylinder and the hanging plate. And is connected to the second spray head, when the cylinder rotates, the second water supply pump supplies the cleaning liquid in the disinfection liquid cavity to the second spray head, and the second spray head flushes and disinfects the inside of the bottle The cylinder drives the inner bottle to be flipped when the mouth of the bottle is turned down, and the disinfection solution flows out of the mouth of the bottle. When the cylinder rotates, the hose and the hose channel move with the cylinder, avoiding The pulling caused the hose to rupture.

The beneficial effect of the present invention is that the present invention can quickly clean the outer surface of the recovered glass bottle and remove old labels. By hot water cleaning, both the bottle body can be sterilized by high temperature and the glass bottle can be heated at the same time. In order to accurately remove the foreign glass in the bottle or the damaged glass bottle in the next infrared detection, the mechanism of subsequent cleaning and disinfection in the bottle is relatively simple, which saves the equipment cost.

### BRIEF DESCRIPTION OF THE DRAWINGS

For ease of description, the present invention is described in detail by the following specific embodiments and the accompanying drawings.

FIG. 1 is a schematic diagram of the overall structure of an automatic disinfection waste glass bottle recycling equipment of the present invention;

FIG. 2 is a cross-sectional view taken along the “A-A” direction of FIG. 1;

FIG. 3 is a cross-sectional view taken along the “B-B” direction of FIG. 1;

FIG. 4 is a cross-sectional view taken along the “C-C” direction of FIG. 1;

FIG. 5 is a cross-sectional view taken along the “D-D” direction of FIG. 1;

FIG. 6 is a cross-sectional view taken along the “E-E” direction in FIG. 3.

### DETAILED DESCRIPTION OF THE INVENTION

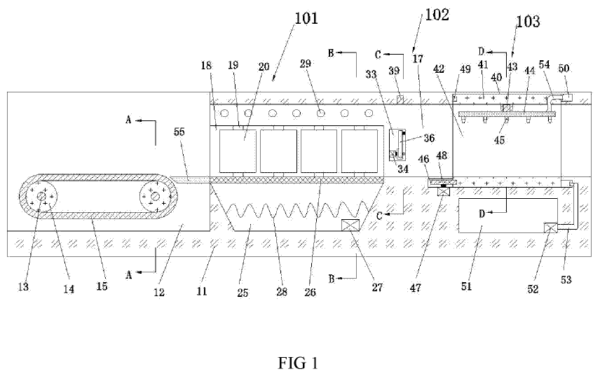

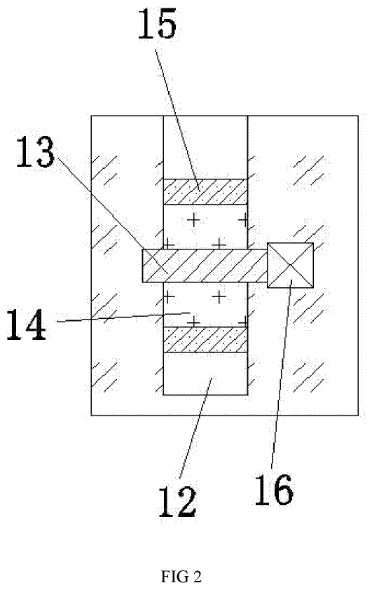

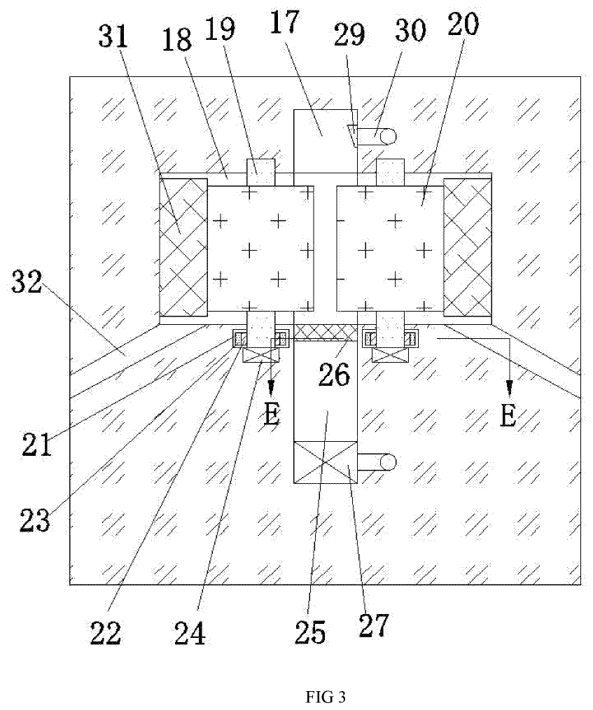

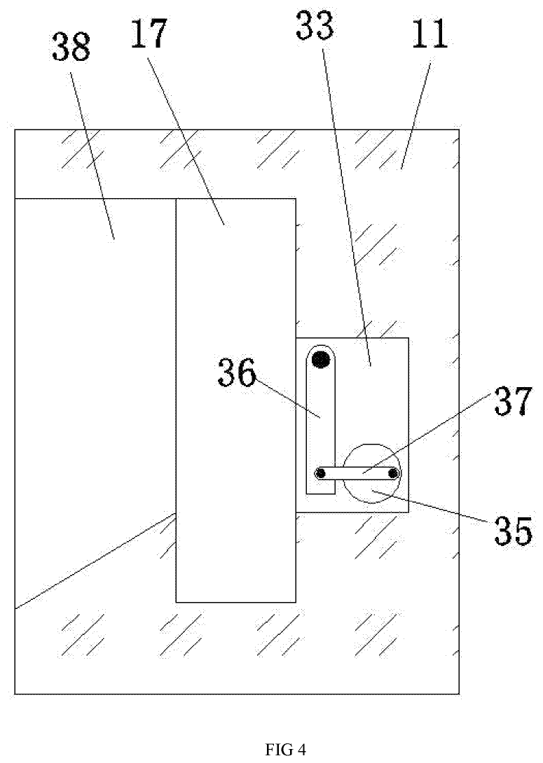

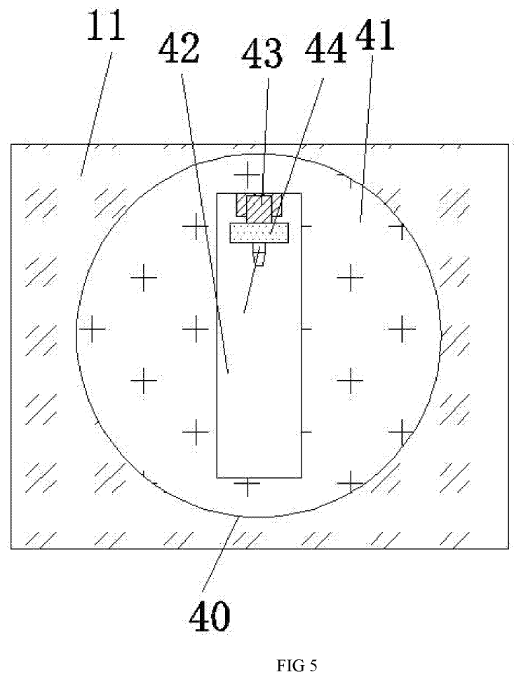

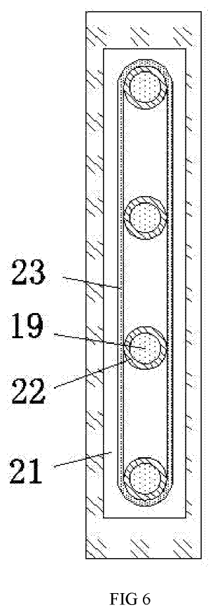

The present invention will be described in detail below with reference to FIGS. 1-6. For convenience of description, the orientation described below is defined as follows: the up-down, left-right, front-back direction described below is consistent with the up-down, left-right, front-back direction of the projection relationship of FIG. 1 itself.

The invention relates to an automatic sterilizable waste glass bottle recycling equipment, which is mainly used for cleaning and disinfection of recycled glass beer bottles, cola bottles, etc. The invention will be further described below with reference to the accompanying drawings of the invention:

An automatic sterilizable waste glass bottle recycling device according to the present invention includes a body 11, and a left side of the upper end of the body 11 is provided with a placement opening 12 that is open upward and leftward, and the right wall of the placement opening 12 communicates A channel 17 opening to the right is provided, and a bottle body scrubbing device 101 is provided on the left side of the channel 17. The bottle body scrubbing device 101 includes a brush roller cavity 18 symmetrically and connected to the left side of the front and rear walls of the channel 17. A rotating shaft 19 is uniformly and rotatably arranged between the upper and lower walls of the brush roller cavity 18, and a brush roller 20 is fixed on the outer surface of the rotating shaft 19, and an end of the brush roller 20 is extended to the channel 17 The label on the surface of the passing glass bottle is cleaned inside. An infrared detection device 102 is provided in the channel 17 and on the right side of the bottle washing device 101. The infrared detection device 102 includes a communication device disposed behind the channel 17. And a bottle pushing cavity 33 on the right side of the brush roller cavity 18, the bottle pushing cavity 33 is provided with a bottle pushing rod 36 whose upper end is rotatably connected to the upper end of the right wall of the bottle pushing cavity 33, The bottle cavity 33 is turned forward to push out the bottle with foreign objects or damage in the bottle, and the right side of the channel 17 is provided An in-bottle scouring device 103 is provided. The in-bottle scouring device 103 includes a chute 40 connected to the upper and lower walls and the front and rear walls of the channel 17, and a cylinder 41 is rotatably provided in the chute 40. A through hole 42 having the same shape as the channel 17 is provided through the left and right of the center. After the glass bottle enters the through hole 42, the bottle flushing device 103 cleans, disinfects and blocks the inside of the bottle in the through hole 42. The bottle on the left side of the bottle flushing device 103 moves to the right.

Beneficially, the lower end of the front and rear walls of the placement port 12 is symmetrically and rotatably provided with a transmission shaft 13. A transmission wheel 14 is fixed on the outer surface of the transmission shaft 13, and a transmission belt 15 is wound around the transmission wheel 14. A first motor 16 is fixed in the rear wall of the placement port 12 and behind the transmission shaft 13 on the right side; the rear end of the transmission shaft 13 on the right side is power-connected to the front end of the first motor 16; On the right wall of the placement opening 12, a receiving plate 55 with an upper end surface located on the same plane as the lower wall of the channel 17 is fixed, the left end of the receiving plate 55 extends to the right side of the conveyor belt 15, and the upper end surface of the receiving plate 55 Slightly lower than the upper end face of the conveyor belt 15.

According to the embodiment, the bottle body scrubbing device 101 is described in detail below. The bottle body scrubbing device 101 includes a transmission cavity 21 provided in the lower wall of the brush roller cavity 18 and below the rotating shaft 19, and the rotating shaft 19 The lower end extends into the transmission cavity 21 and a sprocket 22 is fixedly arranged therebetween. A roller chain 23 is arranged around the sprocket 22. A second motor 24 is fixed below the rotating shaft 19, and the lower end of the rightmost shaft 19 is dynamically connected to the second motor 24.

Beneficially, a water tank 25 is connected to the lower wall of the channel 17, and upper and lower walls of the water tank 25 are fixedly provided with filter plates 26 whose upper end faces are on the same plane as the lower wall of the channel 17, and the lower wall of the water tank 25 is fixedly provided. A first water supply pump 27 is provided, and a first spray head 29 is evenly distributed and fixed on the rear wall of the channel 17 above the brush roller cavity 18, and the first water supply pump 27 and the first spray head 29 communicate with each other. A first water supply channel 30 is provided. A front or rear wall of the brush roller cavity 18 is fixed on a side of the rotating shaft 19 away from the center of symmetry. A scraper 31 is fixed on the lower wall of the brush roller cavity 18 and is on the scraper. A drain port 32 having an opposite opening direction is connected to the lower part 31. The first water supply pump 27 sprays the water in the water tank 25 through the first water supply channel 30 from the first spray head 29 to flush the bottle body. The scraper 31 scrapes off the label washed by the brush roller 20, and the label slides out from the discharge port 32.

According to the embodiment, the infrared detection device 102 is described in detail below. The infrared detection device 102 includes a third motor 34 fixedly disposed at the lower end of the left wall of the bottle pushing cavity 33, and a rotary disk is connected to the front end of the third motor 34. 35. A connecting rod 37 is rotatably provided on a side of the front surface of the turntable 35 remote from the center of the circle, and the other end of the connecting rod 37 is rotatably connected to the lower end of the bottle pushing rod 36.

Advantageously, an electric heating wire 28 is fixed between the left and right walls of the water tank 25 and above the first water pump 27, and an electrical connection is fixed on the upper wall of the channel 17 and above the bottle pushing rod 36. In the infrared generator 39 of the third motor 34, a slideway 38 with an opening forward is provided on the front wall of the channel 17 and in front of the bottle pushing rod 36. The lower wall of the slideway 38 is inclined downward and forward. The rear end is higher than the lower wall of the channel 17, the heating wire 28 heats the water in the water tank 25, and hot water is sprayed from the first nozzle 29 through the heated bottle, and the infrared generator 39 detects the bottle below The bottle with the foreign matter or damage is pushed into the slideway 38 by the bottle pushing rod 36.

According to the embodiment, the in-bottle scouring device 103 will be described in detail below. The in-bottle scouring device 103 includes a hydraulic arm 43 fixedly disposed at the center of the upper wall of the through hole 42, and a hanging plate is fixed at the lower end of the hydraulic arm 43 44. The lower end surface of the hanging plate 44 is evenly distributed and fixed with a second spray head 45. When the hydraulic arm 43 is extended to drive the hanging plate 44 to drop against the bottle mouth, the second spray head 45 is inserted into the bottle mouth.

Beneficially, the left end surface of the cylinder 41 is provided with a tooth slot 49 opening to the left, the lower end of the left wall of the slide slot 40 is connected with a rotary power cavity 46, and a fourth motor is fixed in the lower wall of the rotary power cavity 46 47. An upper end of the fourth motor 47 is power-connected with a gear 48 with a right end meshing with the cogging 49.

Beneficially, a disinfection liquid chamber 51 is provided in the lower wall of the chute 40, and a second water pump 52 is fixed at the lower end of the right wall of the disinfection liquid chamber 51. A second water pump is connected to the right end of the second water pump 52 Channel 53, a right wall of the chute 40 communicates with a semi-circular hose channel 50, and a hose 54 having a lower end connected to the second water supply channel 53 is provided in the hose channel 50, and the hose 54 The upper end passes through the cylinder 41 and the hanging plate 44 and communicates with the second spray head 45. When the cylinder 41 rotates, the second water pump 52 supplies the cleaning liquid in the disinfecting liquid chamber 51. To the second spray head 45, the second spray head 45 scours and disinfects the inside of the bottle. The cylinder 41 drives the inner bottle to flip until the bottle mouth is flushed down. The disinfection solution flows out of the bottle mouth, and the cylinder 41 rotates. At this time, the inside of the hose 54 and the hose passage 50 moves with the cylinder 41 to prevent the hose 54 from being broken due to pulling.

The following describes in detail the use steps of an auto-sterilizable waste glass bottle recycling device in this article with reference to FIGS. 1 to 6:

At the beginning, the bottle pushing lever 36 is in a vertical state, the port 42 is in communication with the channel 17 and is in a vertical state, the hydraulic arm 43 is in a contracted state, and the hanging plate 44 is in an upper limit position.

During operation, the first motor 16 is started to drive the right-hand conveying shaft 13 and the conveying wheel 14 to rotate, and then to drive the conveying belt 15 to rotate. Move right from the conveyor belt 15 to the upper end of the receiving plate 55 and be pushed to the right by the subsequent glass bottle. The glass bottle enters the channel 17, the electric heating wire 28 is heated to heat the water in the water tank 25, and the first water pump 27 is started to move the water in the water tank 25. Hot water is sprayed from the first nozzle 29 through the first water supply channel 30 to wash the bottle body. The second motor 24 is started to drive the rightmost rotating shaft 19 to rotate, and the rightmost sprocket 22 rotates and passes through the roller chain 23 Drive the rotating shaft 19 on the same side to rotate synchronously, and then the brush roller 20 rotates to wash the surface of the glass bottle and brush the label down. The scraper 31 scrapes off the label stuck in the brush roller cavity 18 and slides it out of the discharge port 32. The hot water falling from the bottle body can flow back into the water tank 25 through the filter plate 26. When the glass bottle passes under the infrared generator 39, if the infrared generator 39 detects that there is a foreign object in the bottle or the bottle body is damaged, The third motor 34 starts, and the third motor The machine 34 drives the turntable 35 to rotate once. When the turntable 35 rotates, the bottle pushing lever 36 is driven by the connecting rod 37 to flip forward to push the bottle into the slideway 38 and slide out of the device. When the turntable 35 rotates once, the bottle pushing lever 36 restarts. After returning to the vertical state, the glass bottle continues to move to the right into the port 42. After entering the number of glass bottles corresponding to the second nozzle 45, the hydraulic arm 43 is extended to drive the hanging plate 44 to drop against the bottle mouth. The second water pump 52 is started, and the disinfection solution in the disinfection solution chamber 51 is rinsed and disinfected from the second spray head 45 into the glass bottle through the second water supply channel 53 and the hose 54. At the same time when the second water pump 52 is started, the fourth motor 47 is started to drive the gear 48 Rotating, the gear 48 drives the cylinder 41 to rotate while blocking subsequent glass bottles from entering the port 42. When the bottle is turned down, the disinfection is finished, the disinfection solution flows out of the bottle, the fourth motor 47 is reversed, and then the cylinder 41 After returning to the initial state, the hydraulic arm 43 contracts and drives the hanging plate 44 to rise back to the initial state. The subsequent glass bottles enter the through port 42 and push the sterilized glass bottles to the right to wait for the subsequent process.

The beneficial effect of the present invention is that the present invention can quickly clean the outer surface of the recovered glass bottle and remove old labels. By hot water cleaning, both the bottle body can be sterilized by high temperature and the glass bottle can be heated at the same time. In order to accurately remove the foreign glass in the bottle or the damaged glass bottle in the next infrared detection, the mechanism of subsequent cleaning and disinfection in the bottle is relatively simple, which saves the equipment cost.

In the above manner, those skilled in the art can make various changes according to the working mode within the scope of the present invention.

1. An auto-sterilizable waste glass bottle recycling device includes a machine body; a left side of the upper end surface of the machine body is provided with a placement opening with an upward opening and a leftward opening, and a right wall of the placement opening communicates with a passage opening to the right; a bottle body scrubbing device is provided on the left side of the channel, and the bottle body scrubbing device includes brush roller cavities symmetrically and connected to the left and right sides of the channel; the brush roller cavity is evenly distributed between the upper and lower walls; a rotating shaft is provided on the outer surface of the rotating shaft; a brush roller is fixed on the outer surface of the rotating shaft; the end of the brush roller extends into the channel to clean the label on the surface of the passing glass bottle; an infrared detection device is provided in the channel and on the right side of the bottle body scrubbing device, and the infrared detection device includes a bottle pushing cavity that is connected to the rear wall of the channel and is on the right side of the brush roller cavity; the bottle pushing cavity is provided with a bottle pushing rod that is rotatably connected to the upper end of the right wall of the bottle pushing cavity, and the bottle pushing cavity is turned forward to push out a bottle with a foreign object or a damaged bottle; an in-bottle scouring device is provided on the right side of the channel, and the in-bottle scouring device includes a chute connected to the upper and lower walls and the front and rear walls of the channel; a cylinder is rotatably provided in the chute, and the cylinder the left and right through the center are provided with a port having the same shape as the channel; after the glass bottle enters the port, the bottle flushing device cleans and disinfects the bottle inside the port and blocks the bottle flushing device to the left; the side bottle moves to the right.

2. The recycling device for waste glass bottles that can be automatically sterilized according to claim 1, characterized in that: the lower end of the front and rear walls of the placing port is symmetrically and rotatably provided with a transmission shaft, and the outer surface of the transmission shaft is fixedly provided. There are transmission wheels, and a transmission belt is wound between the transmission wheels; a first motor is fixed in the rear wall of the placing port and on the rear side of the transmission shaft on the right side, and the rear end of the transmission shaft on the right side is dynamically connected to the front end of the first motor; the right wall of the placing mouth is fixedly provided with a splice plate whose upper end surface is on the same plane as the lower wall of the channel, the left end of the splice plate extends to the right side of the conveyor belt, and the end surface of the splice plate is slightly lower than the conveyor belt.

3. The waste glass bottle recycling equipment capable of being automatically sterilized according to claim 1, wherein the bottle body scrubbing device comprises a transmission cavity provided in a lower wall of the brush roller cavity and below the rotating shaft, The lower end of the rotating shaft extends into the transmission cavity and is fixedly provided with a sprocket, and a roller chain is arranged around the sprocket; a second motor is fixed in the lower wall of the transmission cavity and below the rightmost shaft, and the lower end of the rightmost shaft is dynamically connected to the second motor.

4. The recycling device for waste glass bottles that can be automatically sterilized according to claim 1, characterized in that a water tank is connected to the lower wall of the channel, and upper ends of the front and rear walls of the water tank are fixedly provided with upper end surfaces and the lower wall of the channel. A filter plate located on the same plane, the first wall of the water tank is fixedly provided with a first water pump, the rear wall of the channel is evenly distributed over the brush roller cavity, and a first spray head is fixed and fixed, the first water pump and A first water delivery channel is communicated between the first spray heads; a scraper is fixed on the front or rear wall of the brush roller cavity and on the side of the rotating shaft away from the center of symmetry. The lower wall of the brush roller cavity is connected with a discharge opening opposite to the opening below the hanging plate. The first water supply pump sprays the water in the water tank from the first nozzle through the first water supply channel to flush the bottle body, the hanging plate scrapes off the labels washed by the rollers, and the labels are removed from the drain port slides out.

5. The automatic sterilizing waste glass bottle recycling equipment according to claim 1, wherein the infrared detection device comprises a third motor fixedly arranged at the lower end of the left wall of the bottle pushing cavity, and the third motor The front end is powered by a turntable, and a connecting rod is rotatably provided on a side of the front surface of the turntable away from the center of the circle, and the other end of the connecting rod is rotatably connected to the lower end of the bottle pushing rod.

6. The waste glass bottle recycling equipment capable of being automatically sterilized according to claim 4, characterized in that: a heating wire is fixed between the left and right walls of the water tank and above the first water pump; an infrared generator electrically connected to the third motor is fixed on the upper wall of the channel and above the bottle pushing rod; the front wall of the channel is provided with a slideway with an opening forwardly connected in front of the bottle pushing rod, the lower wall of the slideway is inclined forward and downward and the rear end is higher than the lower wall of the channel;
The electric heating wire heats the water in the water tank, and hot water is sprayed from the first spray head to the heated bottle, and the infrared generator detects the bottle below, and the bottle with foreign matter or damage is pushed to the bottle Inside the slide.

7. The recycling device for waste glass bottles that can be automatically sterilized according to claim 1, wherein the flushing device in the bottle comprises a hydraulic arm fixedly arranged at the center of the upper wall of the port, and the lower end of the hydraulic arm is fixedly arranged There is a hanging plate. The lower end surface of the hanging plate is evenly distributed and fixed with a second spray head. When the hydraulic arm is extended to drive the hanging plate to drop against the bottle mouth, the second spray head is inserted into the bottle mouth.

8. The recycling device for waste glass bottles that can be automatically sterilized according to claim 1, characterized in that: the left end surface of the cylinder is provided with a tooth slot opening to the left, and the lower end of the left wall of the slide slot is connected with a rotary power cavity. A fourth motor is fixedly arranged in the lower wall of the rotating power cavity, and a gear at the right end of the fourth motor is meshed with the tooth groove.

9. The recycling device for waste glass bottles that can be automatically sterilized according to claim 1, characterized in that: a disinfection liquid cavity is provided in the lower wall of the chute, and a second delivery is fixed at the lower end of the right wall of the disinfection liquid cavity. Water pump, a second water supply channel is connected to the right end of the second water supply pump, a semi-circular hose channel is connected to the right wall of the chute, and a lower end is connected to the second water supply channel in the hose channel. A hose, the upper end of which passes through the cylinder and the hanging plate and communicates with the second spray head; when the cylinder is rotated, the second water supply pump supplies the cleaning liquid in the disinfection solution chamber to the second spray head, the second spray head flushes and disinfects the inside of the bottle, and the cylinder drives the inner bottle to flip When the flushing is completed when the mouth of the bottle is facing downward, the disinfectant flows out of the mouth of the bottle. When the cylinder rotates, the hose and the hose channel move with the cylinder to prevent the hose from being broken due to pulling.
